(19) United States
(12) Patent Application Publication
(10) Pub. No.: US 2011/0301423 A1
(43) Pub. Date: Dec. 8, 2011

(54) SURGICAL RETRACTOR SYSTEMS

(76) Inventors: Tibor Koros, Moorpark, CA (US); Gabriel Koros, Thousand Oaks, CA (US)

(21) Appl. No.: 13/194,619

(22) Filed: Jul. 29, 2011

Related U.S. Application Data
(63) Continuation-in-part of application No. 29/292,659, filed on Oct. 22, 2007, now Pat. No. D565932.
Application No. 29/292,659 is a continuation-in-part of application No. 12/284,464, filed on Sep. 22, 2008.

(51) Int. Cl. A61B 1/32; G05G 5/06

(52) U.S. Cl. 600/229; 74/536; 600/231

(57) ABSTRACT

Positioning mechanisms can be used with surgical retractor systems to allow a surgeon to easily laterally move, tilt and lengthen each individual retractor blade used in the system. A lateral retraction mechanism provides the necessary actuating forces to laterally move and hold tissue at the surgical site. A retractor blade tilt mechanism can be used to change the angular position of a retractor blade at a surgical site. This tilt mechanism allows the surgeon to further increase the size of the surgical site by tilting one or more of the retractor blades allowing the distal end or toe of each blade to move laterally via a tilting action. A retractor blade holding mechanism allows the surgeon to hold each retractor blade in a fixed position within a blade holder and allows the surgeon to easily change the length of the retractor blade which extends out of the blade holder.

### RELATED APPLICATIONS

This application is a continuation-in-part application of U.S. application Ser. No. 12/284,464 filed on Sep. 22, 2008 which is a continuation-in-part application of U.S. Design application Ser. No. 29/292,659 filed Oct. 22, 2007.

### BACKGROUND OF THE INVENTION

The present inventions generally relate to medical devices for retracting body tissue during a surgical procedure, and more particularly, to retractor blades and their associated mechanisms which can be used to move and maintain tissue to create an exposed surgical field that does not obstruct either visual or physical access to the surgeon. The present invention is particularly directed to mechanisms which can be associated with the retractor blades of a surgical retractor to allow ease in the positioning and lengthening of the working portion of the retractor blades during surgery. The present invention is also directed to manually operable ratchet mechanisms which can be used in a wide variety of devices for various applications for incrementally moving a mechanical member in a linear fashion, such as the retractor blade. In still another aspect of the present invention, a mechanism can be used to easily vary the angular position of the retractor blade once placed within the surgical site.

Various surgical retraction systems have been developed over the years for use in surgical operations which require access to internal organs and bone structures. Surgical retraction systems are used to hold back tissue and expose the area in which the surgical operation is to be performed. Once entry is made into a patient, soft tissue is usually dissected away from other structure, such as a portion of the spine, further exposing the field. However, the exposed field must be maintained using instruments that do not obstruct either visual or physical access to the surgeon. In certain surgeries, for example, the retractor system also needs to be strong enough to overcome the forces exerted by large muscle mass and tissue that has been dissected away from the field of exposure, while maintaining proper visual and manual access to the surgeon.

Many retractor systems having been developed which utilize a set of retractor blades to engage and hold the patient's tissue. For example, in some systems, the retractor blades can be mounted on an independent support frame or a frame that is mounted directly to the operation room table above the patient. The retractor blades can be placed at appropriate positions to retract the patient's tissue and expose the area for the surgical procedure. Due to factors such as the size and location of the surgical site, along with the variations in patient size, the desired exposure is not always directed to the center of the operation site, which many times require the blade retractors to be re-positioned, elevated or pushed down on the margins of the incision. For this reason, each retractor blade includes a mechanism used to maneuver the blade as may be needed to achieve a sufficient surgical work site. Accordingly, it is important that the retractor blade and its associated holding and actuating mechanism are designed for ease in repositioning into the various lateral and angular positions which may be needed by the surgical staff in order to properly expose the surgical site for the particular surgery.

For these reasons, the retractor blade and its associated mechanism should be designed such that it is easy to manipulate and easily re-adjustable to achieve the desire tension necessary to hold back the tissue to expose the area to be surgically treated. It also is important that the mechanism holding the retractor blade does not itself create an obstruction to the surgical site.

What have been needed are improved mechanisms which provide the necessary actuating forces to move and hold back tissue via a retractor blade at a surgical site. Moreover, such mechanisms should be easy to deploy and include the ability to linearly and angularly move the retractor blades, as may be needed, to allow greater flexibility in retractor blade placement at the surgical site. Additionally, such mechanisms should not obstruct access to the surgical site. The present inventions satisfy these and other needs.

### SUMMARY OF THE INVENTION

The present invention provides multi-position locking mechanisms which can be used with surgical retraction systems to provide exposure of an operative site. The present inventions provide sturdy, readily adjustable and easy to use mechanisms which can provide the necessary actuating force needed to retract and hold tissue in place when creating a surgical site. The present inventions are particularly useful when used with a retractor blade system that utilizes a set of movable retractor blades that moves and holds tissue at an exposed surgical site. A surgical retractor system made from these various mechanisms allows the surgeon to easily move, tilt and lengthen each individual retractor blade used in the system.

In one aspect, the present invention relates to a lateral retraction mechanism which provides the necessary actuating forces to laterally move and hold tissue at the surgical site. This lateral retraction mechanism can be associated with a support frame having a large opening which does not obstruct either visual or physical access to the surgeon. In one aspect, the lateral mechanism moves an instrument mounting member and its associated retractor blade in a bi-directional or lateral fashion along the support frame. In another aspect, the mechanism utilizes a rack and pinion assembly attached to the instrument mounting member which moves this member along with its associated retractor blade laterally along the support frame. The mechanism may include a handle, a rotatable shaft and locking components which allow the mechanism to be locked or unlocked by simply pivoting the handle between an upright or downward position. Each retractor blade can be positioned by its own lateral retracting mechanism which allows the surgeon to laterally move the blades as may be needed.

In another aspect, the present invention relates to mechanisms which can be used to change the effective working length of a retractor blade at the surgical site. This particular mechanism, referred to as a retractor blade holding mechanism, is utilized to hold each retractor blade in a fixed position and allows the surgeon to change the effective working length of the retractor blade. The retractor blade itself is not lengthen, but rather, it is the length of the retractor blade which extends from a blade holder associated with the retractor blade holding mechanism that can be re-positioned as needed to obtain the exact length needed for a given surgical procedure and the particular anatomy of the patient. This particular mechanism thus allows the surgeon to easily adjust the working length of the retractor blade which extends out of the distal end of the blade holder. In this regard, the surgeon can manipulate blade holding mechanism to allow a larger or smaller length of the retractor blade to extend out of the blade holder to adjust the effective working length of each retractor blade.

In one particular aspect of the retractor blade holding mechanism, the components include a retractor blade having a plurality of engagable elements formed thereon. These engagable elements can be, for example, notches or grooves formed on a side of the retractor blade. The retractor blade is coupled to a retractor blade holder which includes a structure for slidably receiving the retractor blade. The mechanism includes an arm linkage pivotally coupled to the retractor blade holder, the arm linkage including an engaging member adapted to engage one of the engagable elements of the retractor blade. The pivoting action of the arm linkage allows it to being moved between an engaged position in which the engaging member engages one of the engagable elements of the retractor blade and a disengaged position in which the engaging member disengages from engagement with the engagable element of the retractor blade. When the mechanism is placed in the disengaged position, the surgeon can move the blade within the blade holder to allow the desired length of the blade to extend out of the blade holder. Once the blade has been positioned within the blade holder, the mechanism can be placed back into the engaged position to hold the blade in place. In another particular aspect, the mechanism may include a shaft housed on the retractor blade holder, the shaft including a cam surface which contacts a portion of the arm linkage to pivotally move the arm linkage when the cam surface is moved. The cam surface can be moved, for example, through rotation of the shaft. In this fashion, the arm linkage can be moved into and out of engagement with the blade through the movement of the cam surface.

In another aspect, the present invention relates to retractor blade tilt mechanisms which can be used to change the angular position of a retractor blade at a surgical site. The surgical site created by the retraction of the set of retractor blades can be further increased through the use of a retractor blade tilt mechanism made in accordance with the present invention. It is possible for the surgeon to further increase the size of the surgical site by tilting one or more of the retractor blades allowing the distal end or toe of each blade to move laterally via a tilting action. This tilting of the retractor blade results in the distal end of the blade moving even further laterally resulting in a wider area of retraction at the surgical site. Again, the present invention provides the surgeon with the ability to easily move each retractor blade both laterally and angularly, as may be needed, in order to create the desired visualization needed for the particular surgical procedure. Since each retractor blade can be mounted on its own retractor blade tilt mechanism, the surgeon can tilt one or more of the retractor blades as may be needed to attain the desired angular position.

In one particular aspect, the retractor blade tilt mechanism is associated with a support frame, at least one instrument mounting member mounted for bidirectional movement relative to the support frame, a retractor blade holder pivotally coupled to the instrument mounting member at a first position and a retractor blade coupled to the retractor blade holder. The tilt mechanism includes a pivoting member pivotally mounted at a second position on the instrument mounting member, the pivoting member having a free end which moves when the pivoting member is pivotally moved. The retractor blade holder includes an engaging portion which engages the free end of the pivoting member, the free end of the pivoting member causing both the blade holder and its blade to pivotally move when the free end is moved causing the blade holder to pivotally move when the free end is moved. In another particular aspect, the pivoting member can be pivotally moved via a rotational member, such as a turn screw, which can be rotated to move the free end of the pivoting member between raised and lowered positions. This, in turn, will cause the blade holder to pivotally move relative to the instrument mounting member, causing the retractor blade to be tilted from its initial angular position.

While exemplary embodiments of the inventions are directed to mechanisms used to position retractor blades which form a composite surgical retraction system, although it should be understood that the present inventions could be applicable to other medical device applications, as well as non-medical applications. Additionally, each different mechanism could be a stand-alone positioning mechanism or could be either combined together with the other disclosed mechanisms or incorporated into other retractor systems.

Other features and advantages of the present invention will become more apparent from the following detailed description of the invention when taken in conjunction with the accompanying exemplary drawings.

### BRIEF DESCRIPTION OF THE DRAWINGS

FIG. 1 is a perspective view of a surgical retractor system made in accordance with the present invention which incorporates particular embodiments of a blade holding mechanism for changing the effective working length of the retractor blade, a ratchet mechanism for laterally moving the retractors blades relative to a support frame and a tilt mechanism for adjusting the angular position of retractor blade relative to the support frame.

FIG. 2 is an exploded perspective view which shows the various components used to form the various mechanisms forming the composite surgical retractor system depicted in FIG. 1.

FIG. 3 is a bottom perspective view of the surgical retractor system depicted in FIGS. 1 and 2.

FIG. 4 is a top view of the surgical retractor system depicted in FIGS. 1-3.

FIG. 5 is a bottom view of the surgical retractor system depicted in FIGS. 1-3.

FIG. 6 is a side elevational view of the surgical retractor system depicted in FIGS. 1-3 and taken along lines 6-6 of FIG. 4.

FIG. 7 is a top view showing the lateral ratchet mechanism depicted in FIGS. 1-6 which is used for bi-directional or lateral movement of the retractor blade relative to the support frame.

FIG. 8 is a top view showing the ratchet mechanism depicted in FIG. 7 in which one of the retractor blades has been moved laterally on the support frame.

FIG. 9 is a top view showing the particular embodiment of the ratchet mechanism depicted in FIGS. 7 and 8 in which all of the retractor blades have been moved laterally on the support frame to create an exposed surgical site therebetween.

FIG. 10 is an exploded perspective view of the various components which form the lateral ratchet mechanism depicted in FIGS. 7-9.

FIG. 11A is a side cross-sectional view of the embodiment of the lateral ratchet mechanism depicted in FIGS. 7-10 with the handle in the closed, locked position.

FIG. 11B is a side elevational view of the embodiment of the lateral ratchet mechanism depicted in FIGS. 7-11A with the handle in the closed, locked position.

FIG. 12A is a side cross-sectional view of the embodiment of the ratchet mechanism depicted in FIGS. 7-11B with the handle placed in an open, unlocked position.

FIG. 12B is a side elevational view of the embodiment of the ratchet mechanism depicted in FIGS. 7-12A with the handle placed in the open, unlocked position.

FIG. 13A is a side cross-sectional view of the embodiment of the ratchet mechanism depicted in FIGS. 7-12B in the open, unlocked position showing the teeth of the rotatable detent member in an upper position with respect to the teeth of the fixed or stationary detent member.

FIG. 13B is an elevated side view of the embodiment of the ratchet mechanism depicted in FIGS. 7-13A in the open, unlocked position showing the teeth of the rotatable detent member in an upper position with respect to the teeth of the fixed or stationary detent member.

FIG. 14A is a cross-sectional view showing various components used to form the retractor blade tilt mechanism depicted in FIGS. 1-6

FIG. 14B is an exploded perspective view showing the various components used to form the retractor blade tilt mechanism depicted in FIGS. 1-6.

FIG. 14C is an bottom view showing the various components used to form the retractor blade tilt mechanism depicted in FIGS. 1-6 with the support frame depicted with phantom lines.

FIG. 15 is a side cross-sectional view taken along lines 15-15 of FIG. 4 of the retractor blade tilt mechanism used to adjust the angular position of the retractor blade relative to the support frame.

FIG. 16 is a side cross-sectional view of the retractor blade tilt mechanism depicted in FIG. 15 as the retractor blade has been tilted at an angle from vertical via actuation of the retractor tilt blade mechanism.

FIG. 17 is an exploded perspective view of the retractor blade holding mechanism depicted in FIGS. 1-6.

FIG. 18 is a partial cross-sectional view taken along lines 18-18 of FIG. 4 of the retractor blade holding mechanism depicted in FIGS. 1-4 and 17 as the retractor blade holding mechanism engages the retractor blade.

FIG. 19 is a partial cross-sectional view of the retractor blade holding mechanism depicted in FIG. 18 as the retractor blade holding mechanism is disengaged from the retractor blade to allow the retractor blade to be re-positioned within the blade holder.

FIG. 20 is partial cross-sectional view of the retractor blade holding mechanism depicted in FIGS. 18 and 19 with the retractor blade expanded to its maximum working length or position within the blade holder.

### DETAILED DESCRIPTION OF THE PREFERRED EMBODIMENTS

In one aspect, the present invention relates to positioning mechanisms which can be used to deliver an actuating force that moves and holds, for example, a retractor blade at a surgical site to move and maintain tissue at an exposed surgical field which does not obstruct either visual or physical access to the surgeon. In another aspect, the present invention relates to retractor blade holding mechanisms which can be used to change the effective working length of a retractor blade at the surgical site. In still another aspect, the present invention relates to retractor blade tilt mechanisms which can be used to change the angular position of a retractor blade at a surgical site. For the sake of illustration, the following exemplary embodiments of the inventions are directed to mechanisms used to position retractor blades which form a composite surgical retraction system, although it should be understood that the present inventions could be applicable to other medical device applications, as well as non-medical applications. Additionally, each different mechanism could be a stand alone mechanism and could be either combined together with the other disclosed mechanisms or incorporated into other retractor systems. Further, although the disclosed embodiments are used with retractor blades of an independent support frame, the inventions could also be used with a surgical frame that is mounted directly or indirectly to an operation room table.

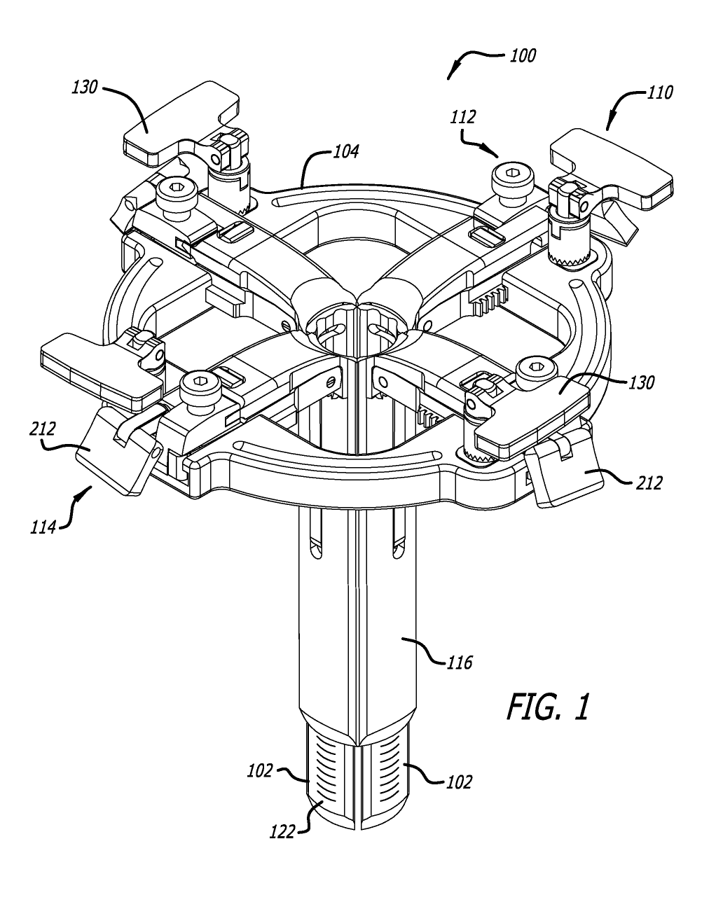

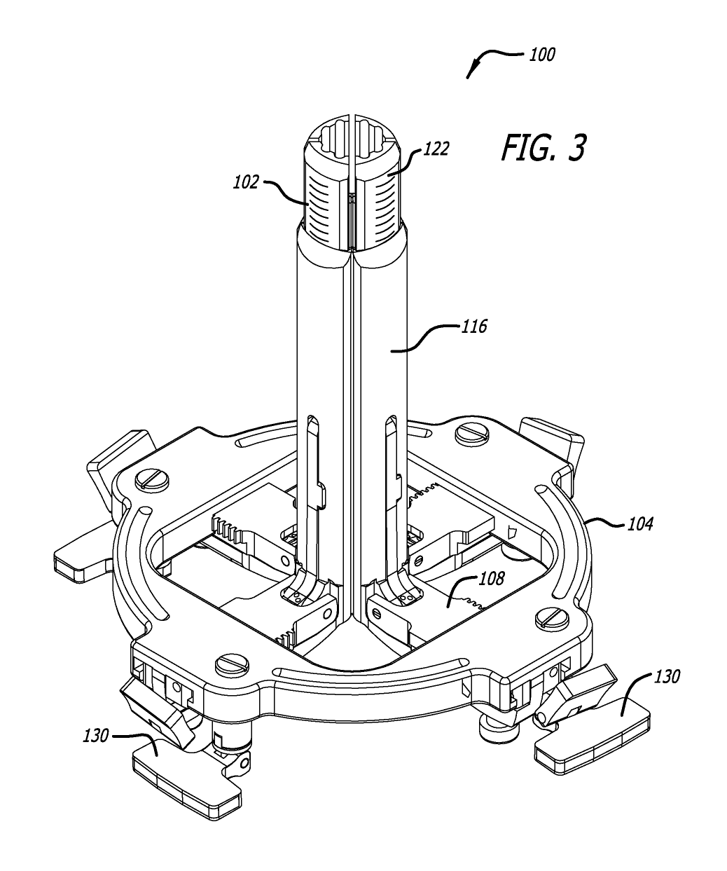

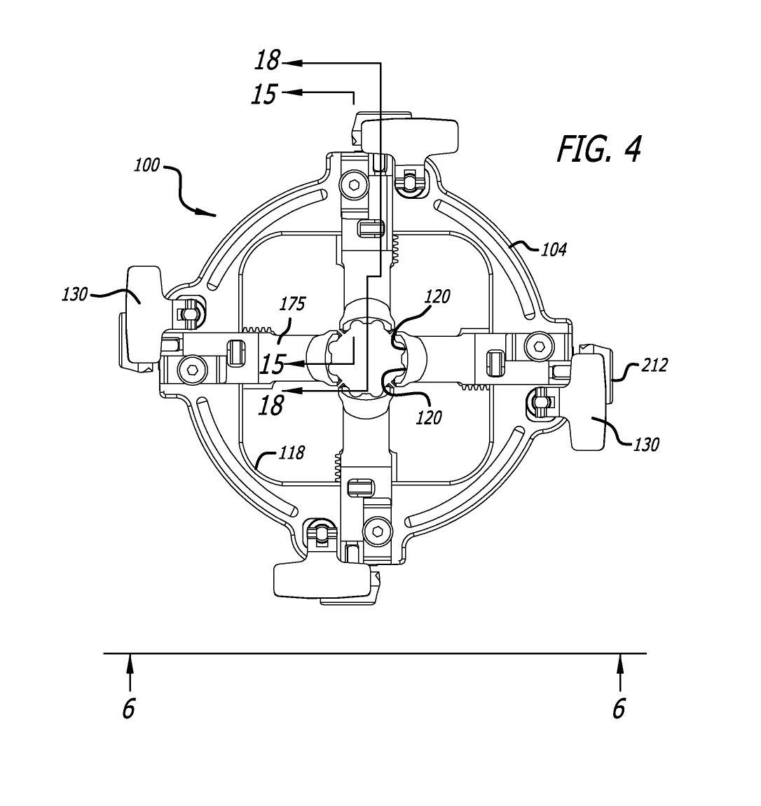

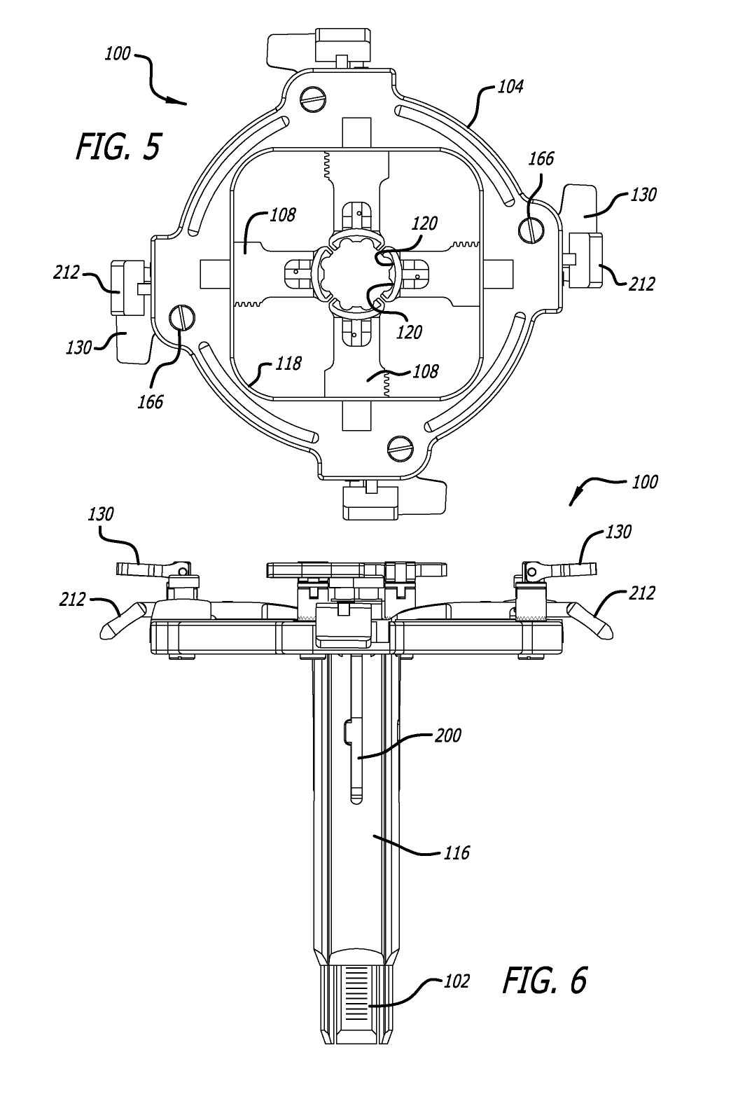

Referring initially to FIGS. 1-6, a composite surgical retractor system 100 is shown which incorporates various positioning mechanisms used to move, tilt and expand the effective working length of a retractor blade 102. In the particular embodiment of the surgical retractor system 100 disclosed herein, a set of four (4) retractor blades 102 are used to move and hold the tissue at a surgical site. It should be appreciated that more or less retractor blades 102 could be used in accordance with the present invention.

The surgical retractor system 100 includes a support frame 104 which supports the various mechanisms used to move, tilt and lengthen each retractor blade 102 of the set. In this regard, the support frame 104 includes a cut or formed channel 106 which receives an instrument mounting member 108 used to mount the various mechanisms made in accordance with the present invention. Each retractor blade 102 used with the surgical retractor system 100 is mounted to an instrument mounting member 108 which allows the retractor blade 102 to be individually tilted, laterally moved, or repositioned with a retractor holder to effectively lengthened the working length of the blade 102. The present inventions allow the surgeon the luxury of positioning and tilting each retractor blade 102, as may be needed, while moving and holding the tissue at the surgical site.

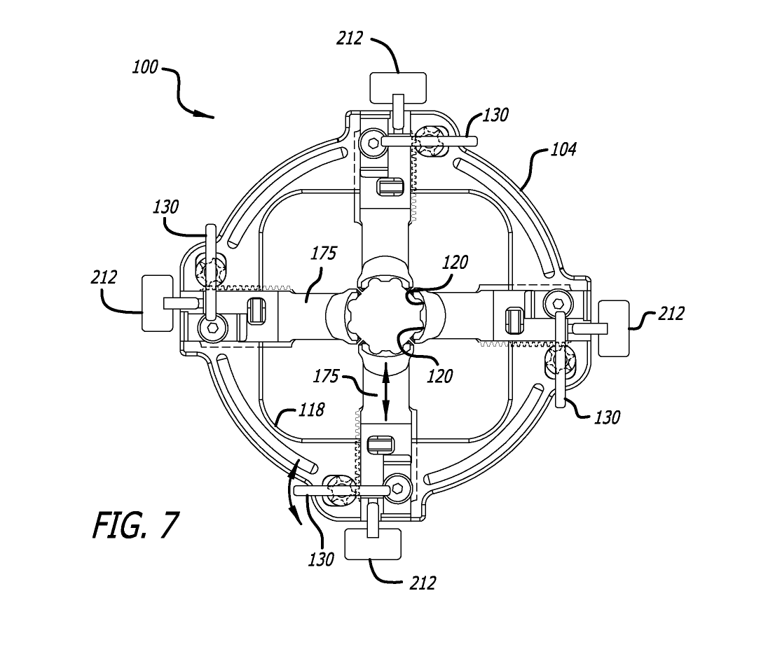

The various mechanisms utilized in accordance with the present invention include a lateral ratchet mechanism 110 which moves the instrument mounting member 108 and its associated retractor blade 102 bi-directionally or laterally along the support frame 104. In this regard, the lateral ratchet mechanism 110 basically moves the instrument mounting member 108 within the channel 106 found on the support frame 104. Each retractor blade 102 will then move laterally with its associated instrument mounting member 108. One particular embodiment of a lateral ratchet mechanism 110 made in accordance with the present invention is disclosed in greater detail in FIGS. 7-13B and in the specification below.

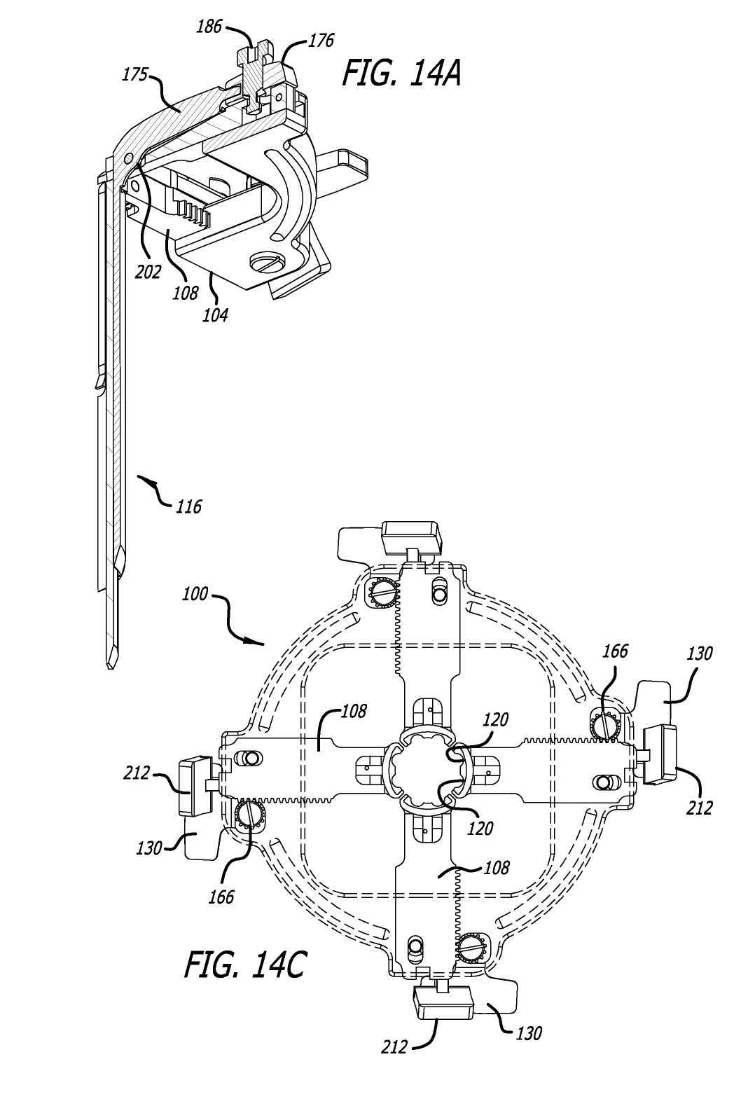

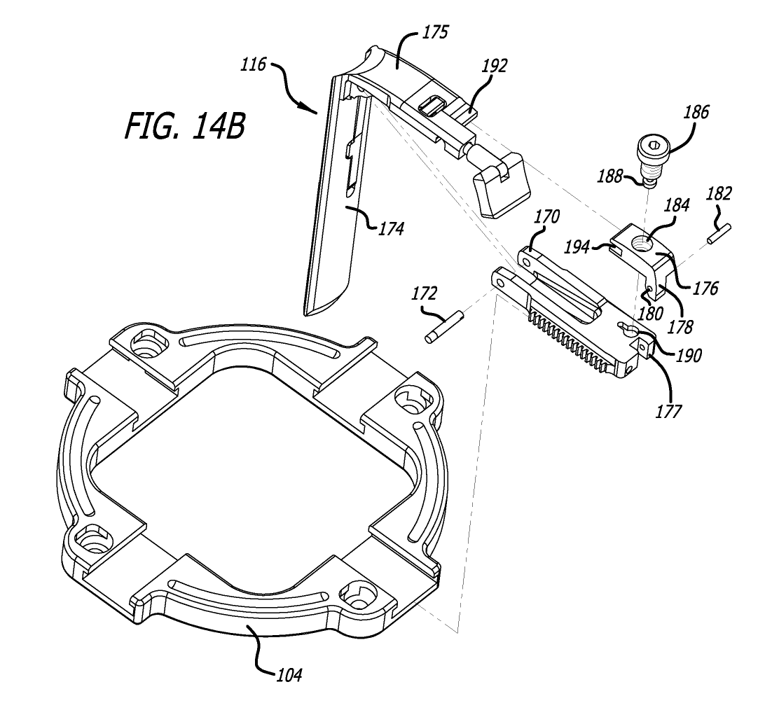

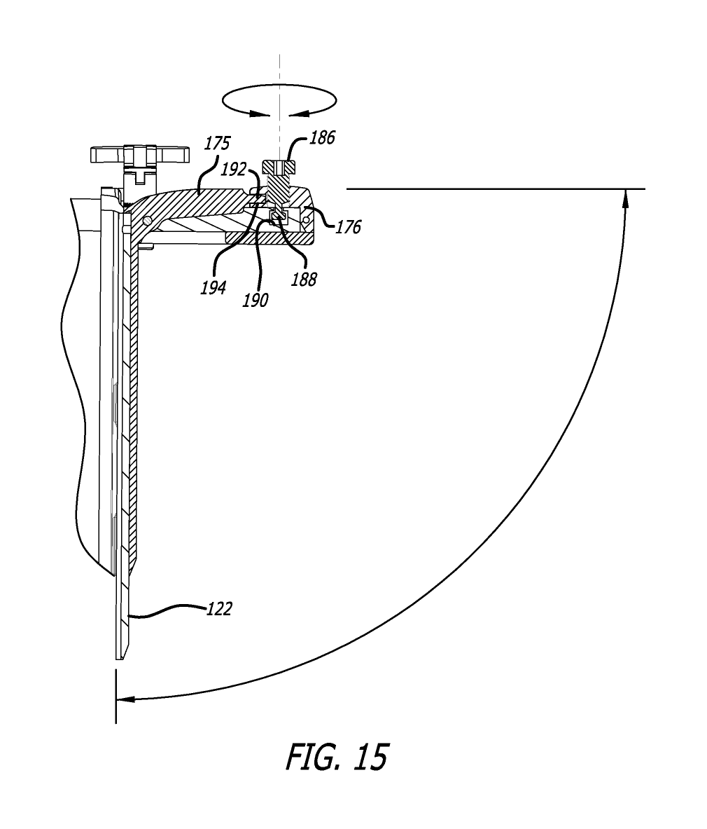

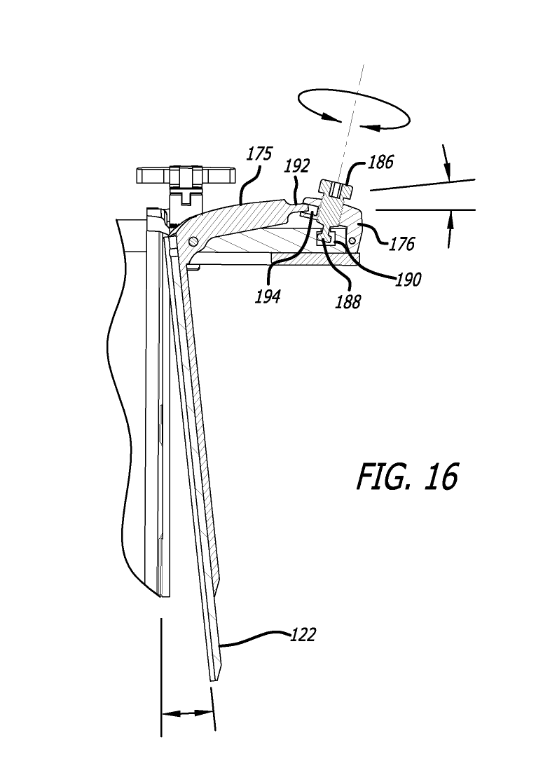

Another mechanism used to tilt the angle of each retractor blade 102 is also mounted to the instrument mounting member 108. This mechanism, herein referred to as a retractor blade tilt mechanism 112, allows the distal end or toe 122 of each retractor blade 102 to be moved away from its initial vertical position, generally shown in FIGS. 1-3. FIGS. 15 and 16 show how a retractor blade 102 can be moved at an angle from the vertical via the manipulation of the retractor blade tilt mechanism 112. One particular embodiment of a retractor blade tilt mechanism 112 made in accordance with the present invention is disclosed in FIGS. 14A-16 and is described in greater detail below.

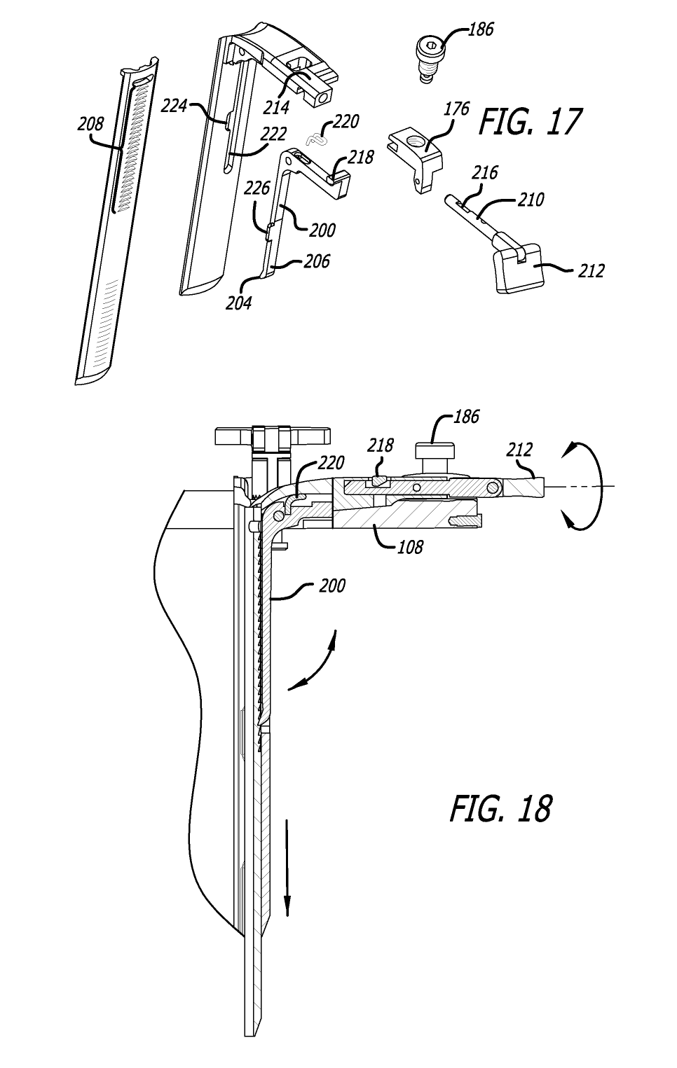

A mechanism utilized to change the effective length of the retractor blade 102 can be mounted on the instrument mounting member 108 as well. This particular mechanism, referred to as a retractor blade holding mechanism 114, is utilized to hold each retractor blade 102 in a fixed position and allows the surgeon to change the effective working length of the retractor blade 102. The retractor blade 102 itself is not lengthen, but rather, it is the length of the retractor blade which extends from a blade holder 116 associated with the retractor blade holding mechanism 114 that can be re-positioned as needed to obtain the exact length needed for a given surgical procedure and the particular anatomy of the patient. This particular mechanism 114 thus allows the surgeon to easily adjust the working length of the retractor blade which extends out of the distal end of the blade holder 116. In this regard, the surgeon can manipulate the blade holding mechanism 114 to allow a larger or smaller length of the retractor blade 102 to extend out of the blade holder 116 to adjust the effective working length of each retractor blade. One particular embodiment of a retractor blade holding mechanism 114 is disclosed in FIGS. 17-20 and is described in greater detail below.

Referring specifically now to FIGS. 7-9, the function of the lateral ratchet mechanism 110 is shown in a series of top views which show the positioning of the set of retractor blades 102 relative to the support frame 104. Initially referring to FIG. 7, the four retractor blades 102 are shown in an initial position in which the retractor blades 102 are very close to one another which depicts a typical blade positioning when the incision made into the patient is relatively small, allowing the closely positioned retractor blades to be placed within this small cavity. As can be seen in FIG. 7, the support frame 104 includes a wide opening 118 which allows for good visualization of the surgical site to the surgeon. Also, as can be seen in FIG. 7, each of the retractor blades 102 has a fluted configuration which includes a pair of channels 120 that can be used to engage a surgical instrument such as a rod or wire (not shown). Such a surgical rod can be positioned in one of the channels 120 and used as a support structure for holding the surgical retractor system 100 within the patient. In this regard, when the surgical retractor system 100 is being utilized, for example, for spinal surgery, the surgical rod could be initially drilled into a portion of the vertebrae of the patient in order to be used as a support structure which will hold the surgical retractor system 100 in an upright position. One of the retractor blades 102 could be utilized as the “support blade” by receiving the surgical rod in one of the channels 120 formed on the retractor blade. Accordingly, the surgical rod and retractor blade will hold the surgical retractor system 100 in an upright position allowing the surgeon to maneuver the other retractor blades 102 as may be needed in order to further expand the surgical incision made in the patient and hold back the patient's tissue.

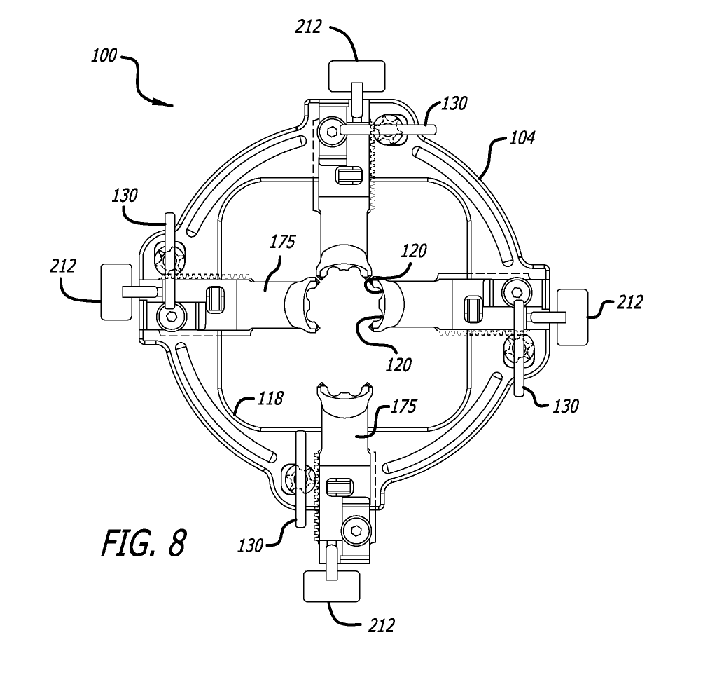

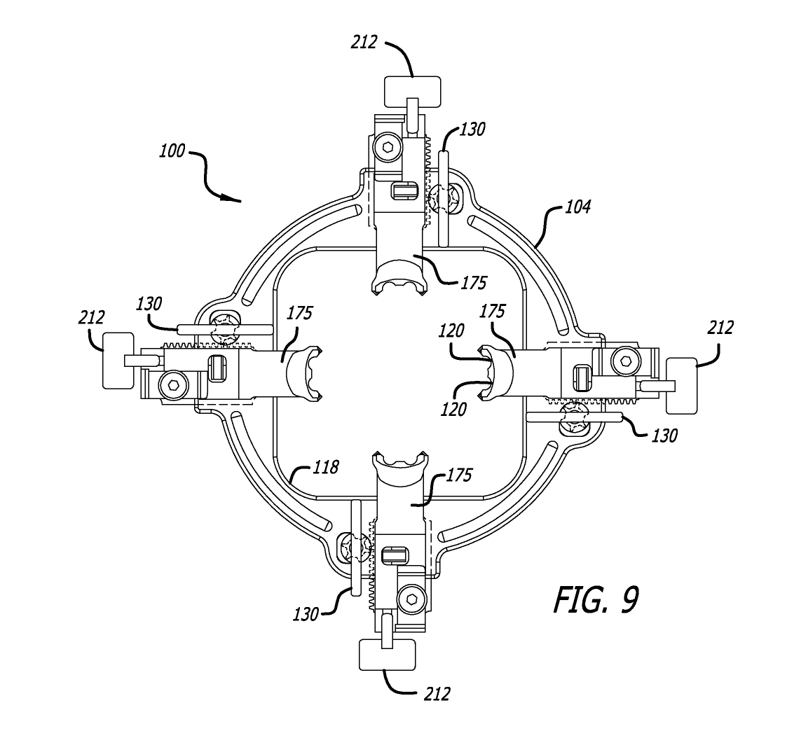

Referring specifically now to FIG. 8, the surgical retractor system 100 is shown with one of the retractor blades 102 moved laterally away from its initial position shown in FIG. 7 which will cause a portion of the patient's tissue to be moved back and held in place in conjunction with the other three retractor blades 102. Each of the other retractor blades can be laterally moved along the support frame 104 to increase the overall size of the surgical site. As can be seen now in FIG. 9, all of four retractor blades 102 have been moved to an expanded laterally position which creates a large surgical area defined by the boundaries of the retractor blades. The retractor blade will thus hold the patient's tissue in place during the surgical procedure. Again, the large opening 118 of the support frame 104 and the large opening created by the four retractor blades 102 provides ample visualization of the surgical site to the surgeon. It should be appreciated that the positioning of each retractor blade 102 can be varied as may be needed by the surgeon in order to achieve the proper size of the surgical site needed for a particular procedure. In this regard, it is possible that the surgeon will not move one or more retractor blades while moving others in order to create the desired visualization needed for the procedure. The fact that each of the retractor is positioned on its own instrument mounting member 108 allows each lateral ratchet mechanism 110 associated with that mounting member 108 to be moved, as may be needed, by the surgeon when creating the surgical site.

The surgical site created by the retraction of the set of retractor blades 102, as is shown in FIG. 9, can be further increased through the use of the retractor blade tilt mechanism 112 made in accordance with the present invention. While FIG. 9 shows the retractor blades 102 in their lateral most position, it is still possible for the surgeon to further increase the size of the surgical site by tilting one or more of the retractor blades 102 allowing the distal end 122 or toe of each blade 102 to move laterally even further than is shown in FIG. 9 to further increase the size of the surgical site. Again, reference is made to FIGS. 15 and 16 which show how the angle of the retractor blade 102 can be tilted, as needed, from a substantially vertical position shown in FIG. 15 to an angular position shown in FIG. 16. This tilting of the retractor blade 102 results in the distal end 122 of the blade 102 moving even further laterally resulting in a wider area of retraction at the surgical site. Again, the present invention provides the surgeon with the ability to easily move each retractor blade both laterally and angularly, as may be needed, in order to create the desired visualization needed for the particular surgical procedure. Since each retractor blade 102 is mounted to its own retractor blade tilt mechanism 112, the surgeon can tilt one or more of the retractor blades 102 as may be needed to the desired angular position.

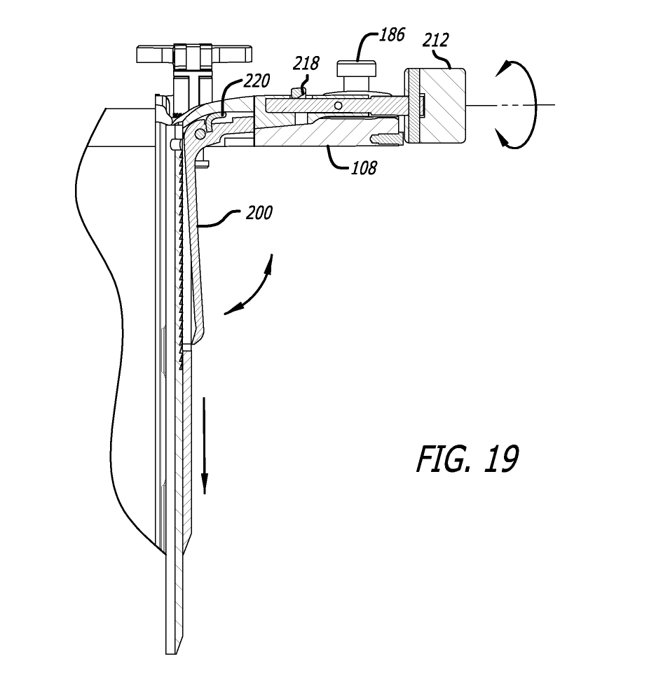

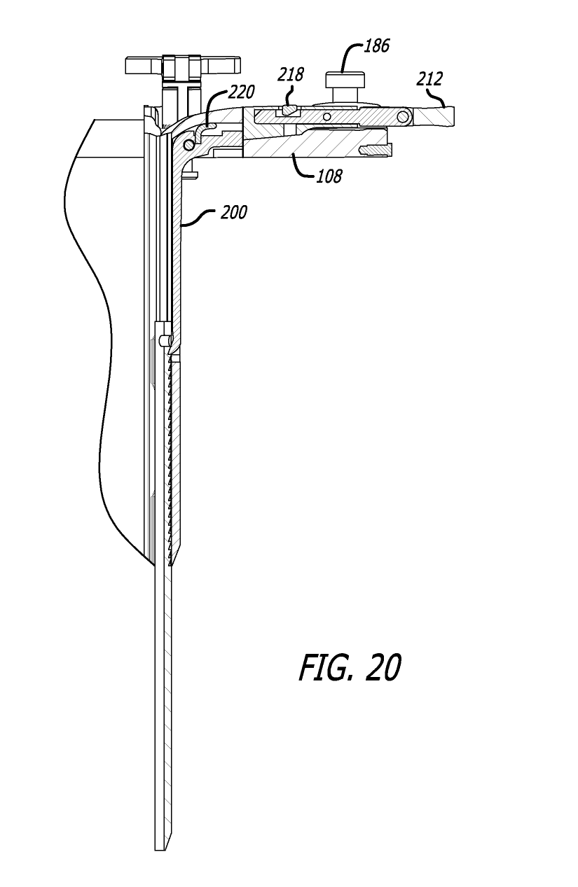

Another benefit of the present invention lies with the blade holding mechanism 114 made in accordance with the present invention which allows the effective working length of each retractor blade to be increased or decreased as may be needed within the surgical site. It should be appreciated that patients come in different sizes and the location of the surgical site will require the use of retractor blades having different sizes. The present invention provides the surgeon with a quick and easy mechanism for adjusting the working length of each retractor blade 102. Referring now to FIGS. 18-20, a particular embodiment of a retractor blade holding mechanism 114 shows how the effective working length each retractor blade 102 can be changed as may be needed by the surgeon. Referring initially to FIG. 18, the retractor blade 102 is shown with its shortest length which can extend out of the blade holder 116. FIG. 19 shows how the blade holding mechanism 114 can be disengaged from the retractor blade 102 to allow the retractor blade 102 to move from its initial position shown in FIG. 18 to its fully extended position shown in FIG. 20. As can be seen in FIG. 20, a greater length of the retractor blade 102 is allowed to extend out of the blade holder 116 which effectively changes the working length of the retractor blade 102. The blade holding mechanism 114 allows the surgeon to easily actuate a handle which disengages the locking mechanism associated with the blade holding mechanism 114 permitting the surgeon to move the desired length of retractor blade 12 out from the blade holder 116.

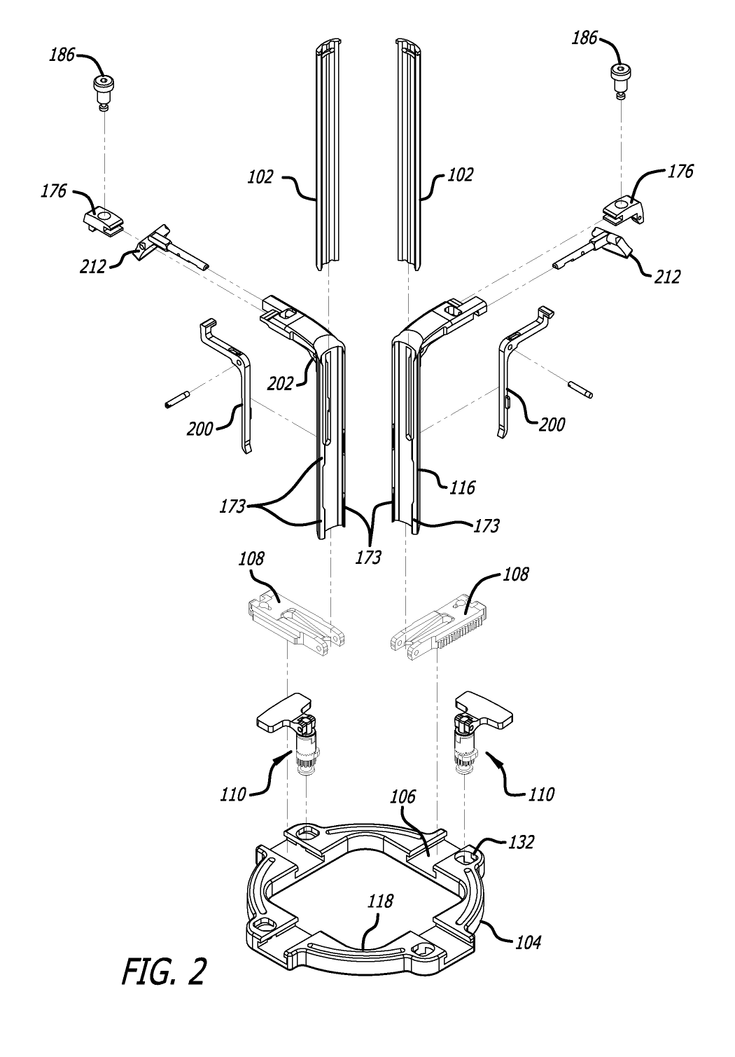

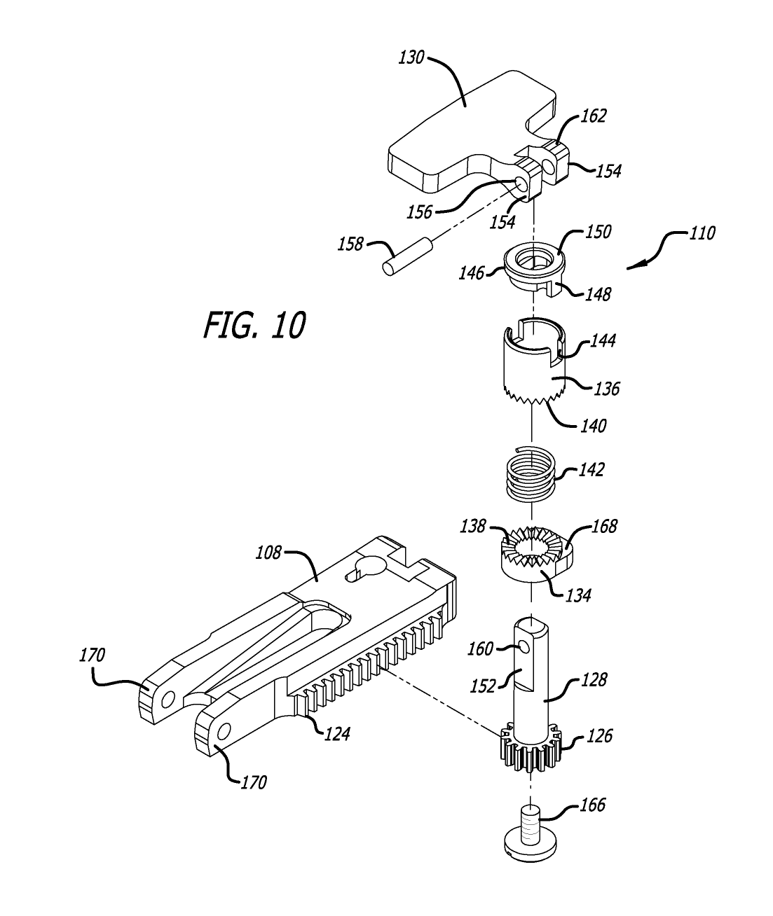

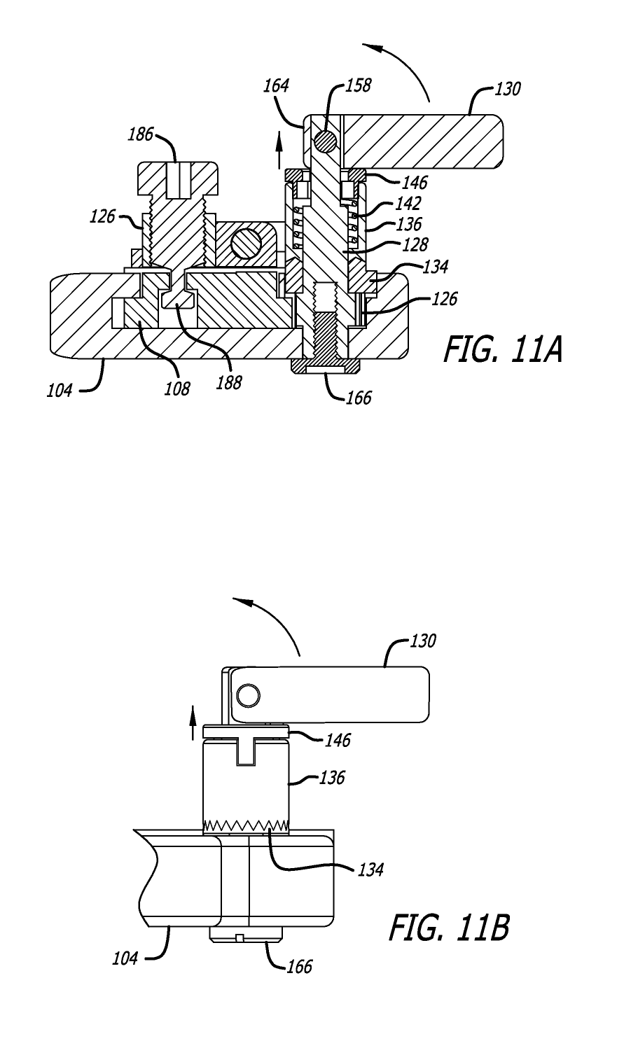

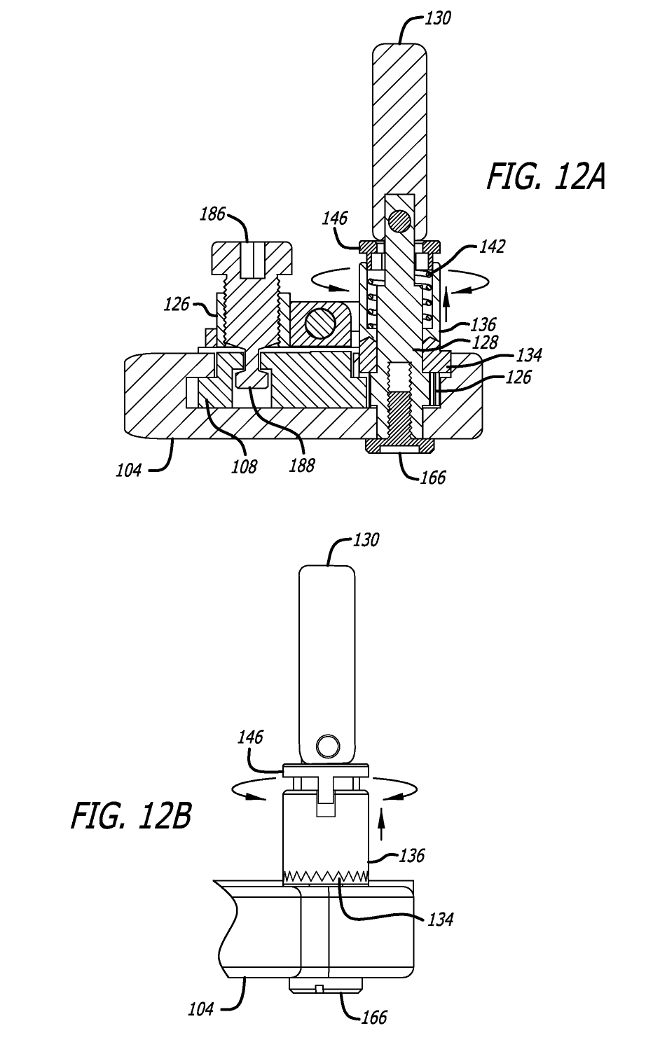

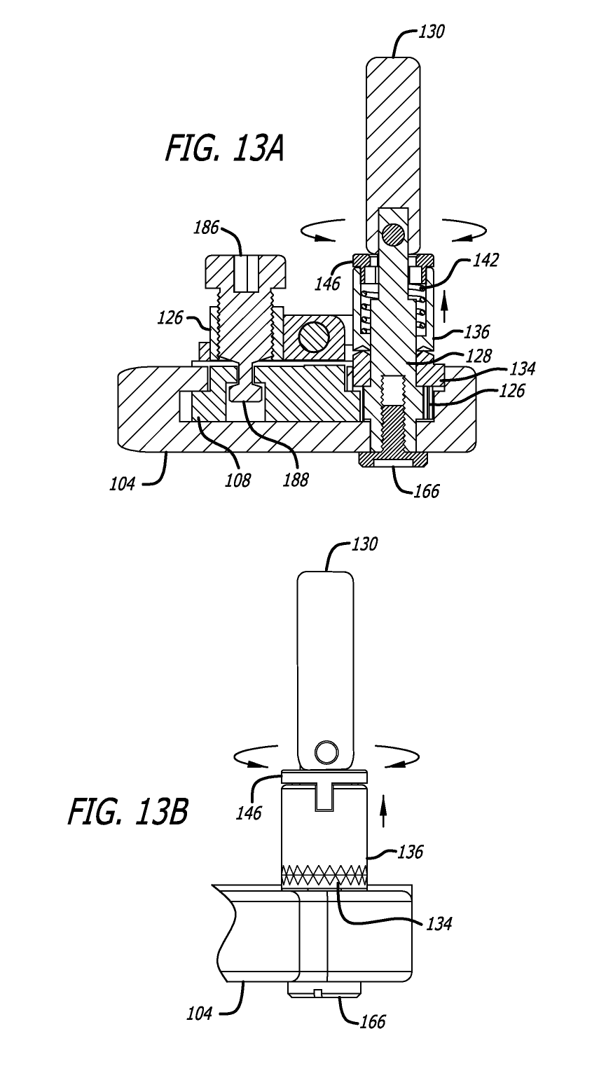

Referring back again to FIGS. 10-13B, a particular embodiment of a lateral ratchet mechanism 110 made in accordance with the present invention is disclosed. In FIG. 10, one particular embodiment of an instrument mounting member 108 is shown which can be used to mount all of the various mechanisms which laterally moves, tilts and expands the retractor blade 102. In FIG. 10, the retractor blade holder 116 used to hold the retractor blade 102 has been removed for ease of visualizing the particular components used with this embodiment of the lateral ratchet mechanism 110. Again, as indicated above, the instrument mounting member 108 is designed to move within a channel 106 formed or cut on the support frame 104. This channel 106 is best seen in FIG. 2 and is further shown in cross-sectional view in FIG. 11A. The lateral ratchet mechanism 110 includes a rack and pinion assembly which allows the instrument mounting member 108 and it's respective retractor blade 102 to move laterally (bi-directionally) along the support frame 104, as is depicted in FIGS. 7-9. As can be seen in FIG. 10, the instrument mounting member 108 includes a toothed rack 124 which engages a pinion 126 that forms part of the rack and pinion assembly. The pinion 126, in turn, is attached to a rotatable shaft 128 which can be rotated by the surgeon via a handle 130 to rotate the pinion 126 to impart bi-directional movement of the rack 124 along with the instrument mounting member 108 and the retractor blade 102. The shaft 128 and the other components making up the lateral ratchet mechanism 110 can be housed within an opening 132 formed on the support frame 104. This opening 132 is configured such that the pinion 126 will engage the teeth of the rack 124 which will move within the channel 106. The surgeon simply rotates the shaft 128 and the pinion 126 by turning the handle 132 when the handle 132 is in the upright, unlocked position shown in FIGS. 12A-13B. Accordingly, the instrument mounting member 108 will move as the rack 124 is being moved via the rotation of the shaft 128 and pinion 126. This movement of the instrument mounting member 108, in turn, moves the associated retractor blade 102 between the positions depicted in FIGS. 7-9.

The particular embodiment of the lateral ratchet mechanism 110 includes a variety of components which allow the position of the instrument mounting member 108 to be placed in a closed or locked position within the channel 106. This will assure the surgeon that the retractor blade 102 will not move once the position of the blade 102 has been set during surgery. Referring again to FIGS. 10 and 11, the lateral ratchet mechanism 110 includes a fixed detent member 134 which engages a rotatable detent member 136. The fixed detent member 134 includes a set of teeth 138 which engages a set of teeth 140 found on the rotatable detent member 136. The rotatable detent member 136 includes a pair of keyways 144 which receive a pair of keys 146 found on a locking member 148. This locking member 148 is disposed over the rotatable detent member 136 to allow the keys 146 to move up and down within the keyways 144 formed on the rotatable detent member 136. This locking member 148 includes a contact surface 150 which can make contact with the handle 130. A biasing element, such as a spring 142, can be disposed between the rotatable detent member 136 and the locking member 148 to apply an upward force on the locking member 148 when the handle 130 is in the unlocked position, as is shown in FIGS. 12A-13B.

The handle 130 includes a pair of ears 154 which extend from the handle 130. Each of these ears 154 includes an opening 156 which receives a pin 158 which locks the handle within another opening 160 formed on the shaft 128. The shaft may include a recessed upper portion which receives these ears 154. Each of the ears 154 has a cam surface 162 which is adapted to move and maintain the locking member 148 in a downward fashion in order to lock the mechanism. As can be seen in FIGS. 11A and 11B, the handle 130 is placed in a downward position preventing it from being rotated. In this position, the cam surface 162 contacts the contact surface 150 of the locking member 148 causing the member 148 to move downward. The keys 146 of this locking member 148 are in turn moved down to abut the bottom of the keyway 144 to help prevent the rotatable detent member 136 from moving upward, as is shown in FIGS. 13A and 13B. Thus, the teeth 140 of the rotatable detent member 136 and the teeth 138 of the fixed detent member 134 will engage themselves to prevent the rotatable detent member from rotating. Hence, the mechanism 110 will be locked to prevent any rotation of the pinion 126 and any lateral movement of the rack 124 and instrument mounting member 108.

Referring now to FIGS. 12A-13B, the manner in which the lateral ratchet mechanism 110 functions to actuate bi-directional movement of the member 108 and retractor blade 102 is illustrated. As can be seen in FIGS. 12A-13B, the handle 130 is placed in its upright position (the unlocked position) which allows the surgeon to grasp the handle 130 to rotate it in order to move the instrument mounting member 108 and retractor blade 102 in the desired position on the support frame 104. The arrows in FIG. 13A shows how the physician can rotate the handle in either direction. FIGS. 12B and 13B show how the locking member 148 extends above the rotatable detent member 136 to allow the rotatable detent member 136 to rotate relative to the fixed detent member 134. Initially, there is a gap between the key 148 and the bottom of the keyway 144. The arrow in FIG. 12B shows that the rotatable detent member 136 can move upward as the handle is rotated. This allows the teeth of the rotatable detent member 136 to disengage from the fixed detent member 134, as is shown in FIG. 13B, allowing the shaft 128 to be rotated. Since the ears 134 of the handle 130 are designed with a flat, front surface 164, the ears do not inhibit the locking member (and key 148) from moving up and down when the handle is being rotated. In the locked position shown in FIGS. 11A and 11B, the cam surface 162 contacts the surface 150 of the locking member 146 to eliminate the gap between the locking member 146 and the rotatable detent member 136. As a result, the key 148 of the locking member 148 and the locking member itself prevents the rotatable detent member 136 from moving upwards to prevent rotation of the shaft 128.

A screw 166 is threaded into the end of the shaft 128 to maintain the lateral ratchet mechanism 110 securely attached to the support frame 104. The detent member 134 may include a structure, such as a lobe 168 which creates an odd shape structure which will fit within the opening 164 formed on the support frame 104 to prevent the fixed detent member 134 from rotating when the shaft 128 is being rotated. In this manner, the fixed detent member 134 will remain stationary during usage. The spring 142 maintains a biasing force on the locking member 146 to allow the rotatable detent member 136 to move upward and downward and rotate as the handle is being rotated.

This particular embodiment of the invention allows incremental movement of the handle as the rotatable detent member 136 is being rotated by the surgeon. When the surgeon wishes to lock the retractor blade 102 and instrument mounting member 108 on the support frame 104 to prevent lateral motion, the handle 130 is simply placed in its downward position shown in FIGS. 11A and 11B. When the surgeon wants to move the retractor blade 102 laterally, he/she merely places the handle 130 back to its unlocked position (FIGS. 12A-13B) and rotates the handle as may be needed.

Referring now to FIGS. 14A-16, one particular embodiment of a retraction blade tilt mechanism 112 is shown in greater detail. In these figures, particularly FIG. 14A, the blade holder 116 is shown as it is pivotally attached to the instrument mounting member 108. Accordingly, the instrument mounting member 108 must include a pair of ears 170 which receive a portion of the blade holder which is held in place by a pin 172. Accordingly, the blade holder 116 will pivotally move in the angular fashion depicted in FIGS. 15 and 16.

The blade holder 116 includes a vertical portion 174 which includes flanges 173 (see FIG. 2) that create a holding region to retain the retractor blade 102 therein. The holding region created by these flanges 173 allows the retractor blade 102 to slide therein. The blade holder 116 further includes an upper portion 175 which extends substantially along the length of the instrument mounting member 108. The tilt mechanism 112 further includes a pivoting member 176 which includes a finger 178 that is pivotally attached to dog ears 177 formed on the instrument mounting member 108 via a pin 182 or other fastening means. The pivoting member 178 includes a threaded opening 184 which receives a turn screw 186. This turn screw 186 is utilized by the surgeon to raise and lower the pivoting member 176 between a lower position (see FIG. 15) and upper position (see FIG. 16). This in turn actuates the blade holder 116 between its substantially vertical position shown in FIG. 15 and its angular position shown in FIG. 16. As can best be seen in FIGS. 15 and 16, this turn screw 186 includes a flange portion 188 which extends from the distal end of the turn screw. This flange portion 188 is designed to fit within a slotted recess 190 formed on the instrument mounting member 108. This flange portion 188 helps to prevent the turn screw 186 from moving from this recess 190 and it also allows the turn screw 186 itself to pivot relative to the instrument mounting member 108 as the pivoting member 146 moves upward as is shown in FIG. 16. A protruding finger 192 extends from the upper portion 175 of the blade holder 116 and is designed to slidingly engage a recess 194 formed on the pivoting member 176. As can be seen FIG. 16, as the turn screw 186 is rotated, the turn screw 186 will move the pivoting member 176 upright which in turn causes the upper portion 175 of the retractor blade holder 116 to move upward as well causing the blade holder 116 to pivot on the instrument mounting member 108 to change the angulation of the vertical portion of the blade holder 116 and the retractor blade 102 itself. The projecting finger 192 and the recess 194 formed on the pivoting member 176 engage each other is a sliding fashion to create an articulation region formed between the two pivot points on the instrument mounting member 108. Accordingly, when the surgeon needs to change the angular position of the retractor blade 102, he/she simply rotates the turn screw 186 as may be needed to achieve the desired angular position of the blade.

It is noted that while a thread screw 186 is utilized in conjunction with the present embodiment, a handle such as those shown in the other embodiments could likewise be used. Still other ways of actuating this pivoting member 176 between the upward and downward positions could be used in accordance with the present invention. The present invention thus creates a compact mechanism which can be easily fitted on a suitable instrument mounting member 108 to provide the physician with a simple and easy way to achieve the necessary angulation of the retractor blade.

Referring now to FIGS. 17-20, a particular embodiment of the blade holding mechanism 114 is disclosed. As can best be seen in FIG. 16, the blade holding mechanism 114 includes a number of components which allow the retractor blade 102 to move to different positions within the blade holder 116. The blade holding mechanism 114 includes an arm linkage 200 which is also pivotally attached to the blade holder 116 via a pair of dog ears 202 formed on the blade holder 116. The arm linkage 200 includes an engaging member 204 which is designed to engage a surface of the retractable blade 102 in order to hold the retractor blade 102 in a locked position on the blade holder 116. In the particular embodiment disclosed herein, the engaging member 204 is shown as an engaging finger formed at one of the ends of the arm linkage 200. This engaging member 204 is found on a vertical portion 206 of the arm linkage 200. The engaging member 204 is designed to fit within a plurality of recesses or notches 208 (engagable elements) formed on one side of the retractor blade 102. This engaging member 204 can be initially placed in one of the recesses 208 to hold the retractor blade 102 in place within the blade holder 116 as is shown in the cross-sectional view of FIG. 18. Thereafter, when the surgeon wishes to extend the working length of the retractable blade 102, the engaging member 204 can be pivoted away from the recess 208 which allows the retractor blade 102 to slide within the vertical portion 174 of the blade holder 116, as is shown in the cross-sectional view of FIG. 19. The engaging member 204 can then be brought back into engagement with one of the other recesses 208 formed along the length of the retractable blade 102 to allow more or less of the retractor blade 102 to extend out of the blade holder 116. FIG. 20 depicts how the engaging member 204 of the arm linkage 200 can be pivoted back into in an engaging relationship one of the recesses 208 formed on the retractable blade 102. As can be seen in FIG. 20, the length of the blade 102 which extends out of the vertical portion 176 of the blade holder 116 is much longer than the length shown in the initial position shown in FIG. 18. Thus, the simple pivoting of the arm linkage 200 to move the engaging member 204 into and out of engagement with the one or more of the recesses 208 formed on the retractor blade 102 allows the surgeon the attain proper working length of retractor blade 102 needed for the desired surgical procedure.

The blade holding mechanism 114 includes a mechanism for moving the arm linkage 200 into and out of engagement with the recesses 208 formed on the retractor blade 102. A shaft 210 is connected at one end to a handle 212 that can be manipulated by the surgeon to move the arm linkage 200. This shaft 210 is placed within a housing portion 214 formed on the upper portion 175 of the blade holder 116. The shaft 210 includes a cam surface 216 which comes in contact with a projecting finger 218 forming a portion of the arm linkage 200. As can be seen in FIG. 18, the projecting finger 218 comes in contact with the cam surface 216 of the shaft 210 and will move upward as the shaft 210 and handle 212 are rotated the surgeon. Accordingly, the cam surface 216 of the shaft 210 moves the finger 218 of the arm linkage in an upward or downward motion when the shaft 210 is being rotated via the handle 212. FIG. 19 shows how the cam surface 216 of the shaft 210 moves the finger 216 upward which in turn causes the arm linkage 200 to pivot, causing the engaging member 204 to come out of contact with the recess 208 formed on the retractor blade 102. The disengaged position, shown in FIG. 19, allows the surgeon to move slide the retractor blade 102 within the vertical portion 173 of the blade holder 116 to the desired position. The handle 212 can then be rotated back into the engaged position to lock the blade 102 in place on the blade holder 116.

The blade holding mechanism 114 also includes a spring clip 220 which provides a biasing force to maintain the arm linkage 200 in the downward engaged position, as shown in FIGS. 18 and 20, to maintain the engaging member 204 engaged with one of the recesses 208 formed on the retractor blade 102. The surgeon can thus rotate the shaft 210 via the handle 212 in order to move the engaging member 204 into and out of engagement with the recesses 208 found on the retractor blade 102.

As can be seen in FIG. 16, the vertical portion 174 of the blade holder 116 includes a slot 222 formed therein which houses the vertical portion 206 of the arm linkage 200 when the engaging member 204 is engaged with one of the recesses 208 of the retractor blade 102. The vertical portion 174 of the blade holder 116 also includes a small recess portion 224 designed to act as a stop abutment which engages and abuts against a flange 226 formed on the arm linkage 200 to inhibit any additional forward movement. In this regard, the biasing force of the spring clip 220 will push the vertical portion 206 of the arm linkage 200 through slot 222 with the flange 226 eventually contacting the recessed portion 224. The engaging member 204 will be in a proper position to engage one of the recesses 208 formed on the retractor blade 102 when placed in the engaged positioned. Thus, if the spring clip 220 provides too great of a biasing force on the arm linkage 200, the flange 226 and recess 224 will prevent any further forward motion of the arm linkage 200.

As can be seen in the drawings, each of the retractor blades 102 can have position markers to show the length of blade portion extending out of the blade holder 116. While notches or grooves are utilized as engagable elements formed on the retractor blade, it is understood that other engagable elements could be used as well. The support frame and the various mechanisms can be made utilizing suitable biocompatible materials such as stainless steel and titanium. The retractor blades also can be made from stainless steel, titanium or other commonly used biocompatible materials. Preferably, the materials which are selected should be susceptible to sterilization using such methods as autoclaving.

While the invention has been illustrated and described herein, in terms of its use with surgical retractor systems, it will be apparent to those skilled in the art that the device can take on a number of different forms and a number of different applications, both medical and non-medical. Other modifications and improvements may be made without departing from the scope of the invention.

What is claimed is:

1. A ratchet mechanism, comprising:
a support frame;
an elongate rack mounted for bidirectional movement relative to the support frame;
a shaft mounted on the support frame for axial rotation;
a handle coupled to the shaft for axially rotating the shaft;
a pinion carried by the shaft configured for engagement with the rack;
the handle mounted for pivotal movement around a pivot axis oriented substantially perpendicular to the shaft between a locked position and an unlocked position; and
a locking member in contact relationship with the handle for inhibiting rotation of the shaft when the handle is in the locked position.

2. The ratchet mechanism of claim 1, further including a rotatable detent member coupled to the shaft for rotation therewith, the rotatable detent member being movable between a first position and a second position when the handle is in the unlocked position; wherein when the handle is in the locked position the locking member engages the rotatable detent member to inhibit the rotatable detent member from moving between the first and second positions to prevent rotation of the shaft.

3. The ratchet mechanism of claim 2 further including:
a fixed detent member located on the frame, the rotatable detent member being rotatable relative to the fixed detent member, the rotatable detent member and the fixed detent member being configured to cooperatively define a series of discrete detent positions.

4. The ratchet mechanism of claim 2, wherein the handle includes a cam surface configured to move the locking member between the locked and unlocked positions.

5. The ratchet mechanism of claim 2 further including a spring for biasing the locking member away from the rotatable detent member.

6. The ratchet mechanism of claim 2, wherein the shaft is mounted for rotation relative to the elongate shaft.

7. The ratchet mechanism of claim 1, wherein the handle member is mounted for pivotal movement around a pivot axis oriented substantially perpendicular to the bidirectional movement of the rack.

8. The ratchet mechanism of claim 1, further including an instrument mounting member attached to the elongate rack, a blade holder attached to the instrument mounting member and a retractor blade mounted on the blade holder.

9. A tilt mechanism for changing the angular position of a retractor blade, comprising:
a support frame;
at least one instrument mounting member mounted for bidirectional movement relative to the support frame;
a retractor blade holder pivotally coupled to the instrument mounting member at a first position;
a pivoting member pivotally mounted at a second position on the instrument mounting member, the pivoting member having a free end which moves when the pivoting member is pivotally moved; wherein the retractor blade holder has an engaging portion which engages the free end of the pivoting member, the free end of the pivoting member causing the blade holder to pivotally move when the free end is moved; and
a retractor blade coupled to the retractor blade holder.

10. The tilt mechanism of claim 9, wherein the engaging portion of the retractor blade holder slidably engages the free end of the pivoting member.

11. The tilt mechanism of claim 9, further including a rotational member associated with the pivoting member to cause the pivoting member to pivot when the rotational member is rotated.

12. The tilt mechanism of claim 9, wherein one of the pivoting member or engaging portion of the retractor blade holder includes a slot and the other of the pivoting member or engaging portion of the retractor blade holder includes an extending finger which extends into the slot and is in sliding engagement within the slot.

13. The tilt mechanism of claim 9, wherein the retractor blade is slidable relative to the retractor blade holder to allow the position at which the retractor blade is held within the retractor blade holder to be varied.

14. The tilt mechanism of claim 9, further including a blade positioning mechanism pivotally mounted on the instrument mounting member and associated with the retractor blade holder for engaging the retractor blade.

15. The tilt mechanism of claim 9, wherein a portion of the blade positioning mechanism is pivotally mounted to the retractor blade holder.

16. The tilt mechanism of claim 11, wherein the pivoting member includes a threaded opening which receives the rotational member, the instrument mounting member includes a recess and the rotational member includes a flanged portion which extends into the recess.

17. A surgical retractor system, comprising:
a retractor blade including a plurality of engagable elements formed thereon;
a retractor blade holder including a structure for slidably receiving the retractor blade; and
an arm linkage pivotally coupled to the retractor blade holder, the arm linkage including an engaging member adapted to engage one of the engagable elements of the retractor blade, the arm linkage being movable between an engaged position in which the engaging member engages one of the engagable elements of the retractor blade and a disengaged position in which the engaging member disengages from engagement with the engagable element of the retractor blade.

18. The surgical retractor system of claim 17, further including a shaft housed on the retractor blade holder, the shaft including a cam surface which contacts a portion of the arm linkage to pivotally move the arm linkage when the cam surface is moved.

19. The surgical retractor system of claim 18, wherein the cam surface is moved through rotation of the shaft.

20. The surgical retractor system of claim 19, further including a biasing member coupled to the arm linkage to bias the arm linkage in the engaged position.

21. The surgical retractor system of claim 17, wherein a portion of the retractor blade holder includes a slot for receiving a portion of the arm linkage when the arm linkage is in the engaged position.

22. The surgical retractor system of claim 21, wherein the retractor blade holder includes a recess and the arm linkage includes an abutting element that contacts the recess when the arm linkage is in the engaged position.

23. The surgical retractor system of claim 18, wherein the arm linkage includes a projecting finger which contacts the cam surface of the shaft and is moved via movement of the cam surface.

24. A surgical retractor system, comprising:
(a) a support frame;
(b) at least one instrument mounting member mounted for bi-directional movement relative to the support frame;
(c) a retractor blade holder coupled to the instrument mounting assembly;
(d) a retractor blade coupled to the retractor blade holder;
(e) a rack and pinion assembly associated with the instrument mounting member for moving the instrument mounting member bi-directionally relative to the support frame;
(f) a tilt mechanism mounted to the instrument mounting member for pivoting the retractor blade holder relative to the support frame; and
(g) a mechanism associated with the retractor blade holder for engaging the retractor blade on the retractor blade and holding the retractor blade in a first position and for disengaging from the retractor blade to allow the retractor blade to be slidable moved to a second position on the retractor blade holder.
